(19) United States
(12) Patent Application Publication
(10) Pub. No.: US 2007/0012101 A1
(43) Pub. Date: Jan. 18, 2007

(54) Method for depicting structures within volume data sets

(76) Inventors: Stefan Rottger, Nurnberg (DE); Marc Stammlnger, Erlangen (DE); Michael Bauer, Erlangen (DE)

Correspondence Address: DENNISON, SCHULTZ & MACDONALD, 1727 KING STREET, SUITE 105, ALEXANDRIA, VA, 22314, US

(21) Appl. No.: 11/443,459

(22) Filed: May 31, 2006

(30) Foreign Application Priority Data
May 31, 2005 (DE) 10 2005 024 949 3

(51) Int. Cl. G01W 1/00; A61B 5/05

(52) U.S. Cl. 73/170.24; 600/420

(57) ABSTRACT

In a method for depicting structures within volume data sets in accordance with the invention, each voxel is therefore allocated a colour and opacity by means of an allocation instruction in dependence upon the scalar values of the voxels. For each scalar value the spatial position of the voxels with this scalar value is determined and from the position coordinates the colour and opacity value of the allocation instruction at the site with this scalar value is determined. The combination of the spatial voxel positions and the scalar values permits targeted depiction of features of the structures being investigated, which can be distinguished by the allocated opacity values.

The invention relates to a method for depicting structures within volume data sets, wherein each voxel is allocated a colour and opacity by means of an allocation instruction in dependence upon the scalar values.

Cross-sections are most commonly used to depict data which are which are widely used in medicine and are acquired e.g. by computer tomography, i.e. CT scanners. However, in order to interpret the data, detection of the spatial relationship and therefore spatial display of the structures investigated are necessary. The corresponding three-dimensional imaging methods are designated as volume visualisation (volume rendering technique). As a result, a two-dimensional image consisting of pixels is depicted on a display unit, wherein each pixel is allocated a colour which is determined from the scalar values of voxels of the volume data set.

So-called transfer functions are typically used to allocate visual properties, e.g. specific colours and opacities, to the voxels or their scalar values for subsequent depiction. The selection of the most suitable transfer function, i.e. suitable colours and opacities, is important in order to find certain features in the volume data set, which are to be depicted. The selection of the transfer function is made empirically using histograrns, i.e. graphical depictions, which reproduce the statistical frequency of the scalar values in a data set and therefore depict the distribution of these values. However, it can also be made freely. The frequency distribution and the frequency values provide the person skilled in the art with information about structural features. The selection of the depiction parameters for depicting the structures or objects, which the procedure of selecting or setting the transfer function represents, requires great experience and is time-consuming.

One problem with volume visualisation, in particular three-dimensional reconstruction from two-dimensional cross-sections, is that the implementation of the reconstruction and the operation of the apparatus required for this purpose need to be uncomplicated and effective. It is important to find specific features (e.g. grey value, spatial position, gradient corresponding to partial structures, such as bones, organs, individual elements such as tumours, etc) in the data sets used and to be able to isolate the associated objects and then make them visible. Otherwise, in the case of more deeply lying structures shadows occur or there is insufficient discrimination of the structures from similar or adjacent structures and also problems at the boundaries of the grey value regions. In order to overcome these problems subsets are usually selected from the volume data sets by means of different segmentation techniques. The segmentation or spatial separation of objects, i.e. a voxel set with the same or similar statistical properties, is very time-consuming.

WO 00/08600 A1 discloses a three-dimensional reconstruction method for structures, which uses the method of segmenting the whole quantity of volume data in order to locate objects in structures. Since the evaluation uses the whole data quantity it is time-consuming and also requires a large computer capacity. The image analysis information thus acquired is used in the planning of three-dimensional radiation therapy treatments.

DE 37 12 639 A1 discloses a method for imaging volume data in which a three-dimensional data set is rendered two-dimensional for display purposes. In so doing, each voxel within the volume is allocated a colour RGB and an opacity A and stored as a three-dimensional data volume RGBA. In so-called partial volume classification, percentages are determined, with respect to which voxels do not consist of a single homogenous material. The imaging of the voxels is based on linking and filtering of the voxel data.

A diagnostic apparatus described in DE 100 52 540 A1 includes means for setting transfer functions which use the frequency distribution of the grey values. This means that statistical properties of the structures being investigated are used for reproduction thereof.

Most of the known structure depiction methods which employ transfer functions use one-dimensional transfer functions. Furthermore, the so-called multi-dimensional transfer functions have been developed which are not only dependent upon the scalar data values (density or grey values) but in which further parameters, i.e. also higher order derivations, e.g. the volume gradient, curvature, etc, are considered, see e.g. Kindlmann and J.W. Durkin, “Semi-Automatic Generation of Transfer Functions for Direct Volume Rendering”, Proc. Visualization Symposium '98, pages 79 to 86, 1998. When using such multi-dimensional transfer functions, features of the structure being investigated can be better located. The multi-dimensional transfer functions also advantageously differ from one-dimensional i.e. standard transfer functions, e.g. in the accentuation of certain properties. However, the evaluation involves a great deal of effort.

The two-dimensional transfer function is frequently used, it is dependent on two scalar values, i.e. the absorption value, density and e.g. the size of the gradient, and can make material boundaries more visible. If, when using this function, e.g. MRI volume structures are made visible, in which it is not possible to distinguish between bone and air, considerable practical know-how is required to set up the transfer function in order, from the distribution of the scalar data values and gradients, to obtain references for the separation of features, e.g. for the display of the tumour and cranium, and to establish the transfer function in a suitable manner. Furthermore, a great deal of time is required to set the appropriate transfer function.

The imaging of volume data sets within the transfer functions i.e. the allocation of a colour for each scalar value of the voxels is known. In the known methods only statistical and not spatial information is used in connection with multi-dimensional transfer functions.

It is the object of the invention to provide a method with which three-dimensional structures can be displayed quickly and simply along with the accentuation of partial structures.

This object is achieved in accordance with the invention by a method having the features of claim 1. A device operating in accordance with the invention is the subject of claim 14. Advantageous developments are the subject of the subordinate claims.

In the case of a method for depicting structures within volume data sets in accordance with the invention each voxel is thus allocated a colour and opacity by means of an allocation instruction in dependence upon the scalar values of the voxels. For each scalar value the spatial position of the voxels with this scalar value is determined, and from the position coordinates the colour and opacity value of the allocation instruction at the site with this scalar value is determined.

The combination of the spatial voxel positions and the scalar values permits targeted depiction of features of the structures being investigated, which can be distinguished by the allocated opacity values. Knowledge of the spatial positions of the voxels, which is linked to the depiction data of the allocation instruction, makes it possible to dispense with statistical evaluations, the use of histograms as an input aid for the selection of voxels from the volume data set, empirical display optimisation or inclusion of additional parameters for location of object structures. The objects can be depicted in a substantially automated manner. The amount of work required to produce the display is correspondingly reduced. The persons responsible for this need only select the display and the modalities of the display of the structures, the desired partial structures or elements on the display or depiction apparatus. By using colour selection and configuration, certain object features can be displayed as desired and others can, in turn, be faded out.

The method in accordance with the invention is carried out as follows: A scalar value is selected. The m voxels which have this scalar value (or a combination of different scalar values) are sought. Their spatial position is observed and e.g. their centroid is calculated. A colour (RGB, A) is then allocated to the m voxels. In this way all entries of the allocation instruction are sifted through, this instruction preferably being a transfer function, as is customary per se in image processing. Instead of the transfer function, however, other location-related depiction instructions are also possible, e.g. those with pre-processing steps, clusterings etc. The allocation of colour to the m voxels leads to colour/centroid groups in the depiction so that it is possible to differentiate between structures. In practice this leads to a number of centroid reproductions in the field of the transfer function, which, by reason of the spatial position e.g. of the centroid, gather at associated points corresponding to the position of the objects.

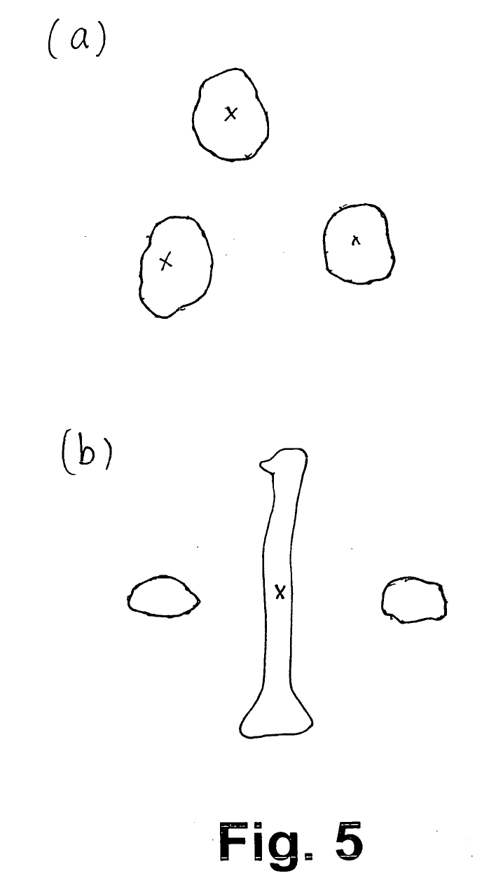

In more detail, when carrying out the method in accordance with the invention the voxels of a volume data set obtained by a CT, MRI or other scanner are allocated n scalar values resulting from the scanning, wherein n is also the dimensional number of the transfer function which can therefore be one-dimensional and also multi-dimensional. For imaging purposes the transfer function is in turn provided to allocate depiction data such as opacity and colour to each scalar value. In this way imaging parameters can be selected with the aid of the scalar values and the properties of the object depiction can be fixed without the voxels having to be accessed directly. By reason of their positional allocation to the scalar values the voxels are called up for image depiction. By means of this functionality, in the first place a clearly smaller amount of data needs to be processed in the case of the selection process not using the positional data of the voxels, and imaging is effected more quickly. It is not necessary to seek feature boundaries since all features are immediately differentiable by looking at the depiction of the transfer function. FIG. 5 (b) shows the case where very remote structures can initially not be separated by reason of the common centroid. In this case object discrimination is possible by using a further scalar value or by consideration of variance. The method in accordance with the invention makes it possible to show partial structures which conventionally, without further measures, could not be resolved within a reasonable amount of time.

However, as also shown by the diagram of FIG. 5 (a) for three structures with the same properties in which the centroid is marked by “x”, the volume visualisation by means of transfer function also has limitations in principle. A transfer function thus usually initially supplies all costal arches only all at once, then, by reason of the common properties, practically the same location is produced in the transfer function and therefore the same colour. A specific costal arch cannot be depicted individually for lack of available local information. However, by means of the invention and using automatic depiction of the associated regions the costal arches can be rapidly and precisely selected by reason of the additional location-coding. This pre-segmentation can now be used to accelerate subsequent segmentation. The procedure is as follows:
1. The features concerned are selected in the transfer function.
2. The selected regions are used as a starting point for the subsequent segmentation.
3. The segmentation is carried out, in that the individual costal arches are allocated to different segments using the spatial relationship between the respective costal arches.
4. A single costal arch can be displayed, in that the associated segment is singled out or selected.

The method in accordance with the invention is advantageously used on CT and MRI volume data sets. Furthermore, it is suitable for use on volume data sets which have been produced using ultrasound, radar and positron emission spectroscopy (PET), etc. There are also other scanning processes to which the method can be applied provided the scans result in a three-dimensional scalar data set representing the properties being investigated, e.g. even in the case of cross-sections, in the depiction of time-dependent data, such as from a plurality of successive scans which have been carried out, in the display of flow ratios, etc.

In the case of material testing the method can show e.g. fissures in specimens and the like, in that two scalar values are checked such as the density and gradient and in the simplest case two colour classes (material, air) are sufficient to indicate a fissure.

CT and MR data sets can also be combined which, in part, supply complementary information, combined knowledge of which can be extremely important. For this purpose the so-called registration (matching) is then carried out, by means of which the CT and MR images are superimposed and a second parameter is introduced so that two scalar values, one from the CT and one from the MR data set, are processed and to this end a two-dimensional transfer function is required. Other or multiple combinations of the data types mentioned or other suitable data types can be used when applying the method in accordance with the invention.

The method in accordance with the invention is also very suitable for evaluating and making visible simulations e.g. of flows, rate distributions, pressure distributions. In a very general way it is used in a supporting capacity in any three-dimensional imaging applications which it also unquestionably simplifies.

The method in accordance with the invention therefore provides an affine transformation method by means of which three-dimensional structures, e.g. in the form of CT or MRI data sets, can be converted into one plane, i.e. a flat depiction. For flat depiction, i.e. as image data values, the associated allocation instruction or imaging function allocates respective positional coordinate-related depiction values to the voxels and their scalar values, which depiction values are expediently the colour and opacity value. However, instead of these values other depiction values can also be used, e.g. instead of the colouring of pixels the pixels can be displayed intermittently in an appropriate cycle.

In one advantageous variation of the method in accordance with the invention, classification is effected according to the spatial position, and each class is allocated a specific colour and/or opacity. All pixels which relate to voxels with the same or similar position or are in a specific fixed spatial relationship to each other are therefore automatically coloured the same or virtually the same or similarly. The centroid can preferably be used as the classification criterion of the spatial position.

A broadening of, or alternative to, the described classification is possible according to the variance, in particular according to the average distance from the said spatial position or the privileged direction, wherein each class is allocated a colour and/or opacity. In the case of vector classification according to the centroid of the voxels and variance of the positions it is also possible to achieve a separation of structures which, by means of the method in accordance with the invention, could still not at first be separated with sufficient clarity, e.g. structures with the same centroid but different form or arrangement. By means of an additional parameter, discrimination by point distribution (variance) can now be carried out.

In such a case of vector classification, which can again take place automatically, the object or class recognition is effected by point accumulation as mentioned. The found object can then be allocated a specific colour for depiction on the display screen. On the other hand, allocation of spatial information can also be effected in such a way that one or a plurality of structure elements can be displayed and the structure element(s) is/are accentuated and/or selected by optical means.

However, it is also possible to carry out segmentation, in that a class of the voxels is selected and the set of the voxels selected is used as a basis for subsequent segmentation.

When depicting the transfer function in accordance with the invention which is used to select the scope of the imaging or of object structures, the brightness is a measure of the presence of many points with the same parameter values. On the other hand, if there are no points with specific parameter values the depiction of the transfer function at the relevant locations is shown in black. In practice, the imaging of the voxels using the parameter depiction in the transfer function leads to colour points which each depict a specific spatial position, e.g. the centroid, of a voxel group with the same parameters. Provision can be made for selecting similar colours for similar objects and likewise different colours for different objects. The depiction of the volume data set can thus automatically be coloured so that structures can be distinguished.

By reason of the rapid implementation of the method in accordance with the invention and of the relatively lower data complexity in determining the object depiction it is possible to use a personal computer (PC) for practical implementation thereof.

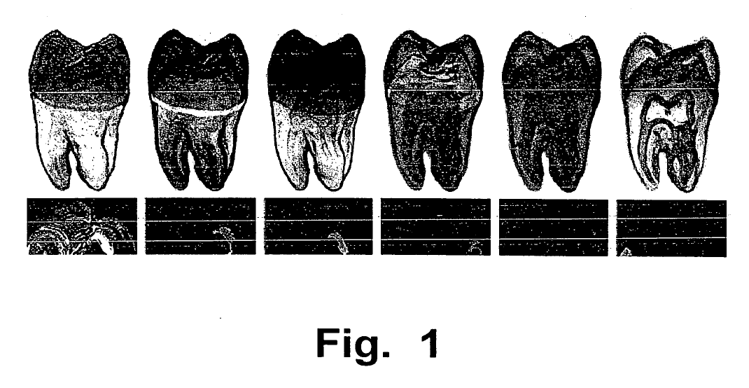

In spatial regions with a low voxel count (e.g. below 5 to 10 voxels) problems in classification can arise by reason of insufficient data, in particular statistical noise may also be produced. If no further measures are taken, the opacity is expediently set to zero for the regions. However, the number of voxels is increased by overscanning so that there are more measuring points in the histogram. In so doing, the number of measuring points is increased (e.g. in the case of overscanning with doubled precision the number of measuring points is increased by a factor of 8). Alternatively or additionally, each voxel can still be entered in a k environment in the histogram depiction (preferably k=1 or 2). In this way the noise can additionally be reduced and the object classification is improved. This technique is carried out in the display in accordance with FIG. 1, which will be referred to again below.

A device for carrying out the method in accordance with the invention comprises an apparatus which allocates a colour and opacity to each voxel by means of an allocation instruction, e.g. transfer function, in dependence upon the scalar values, an apparatus which, for each scalar value, determines the spatial position of the voxels with this scalar value, and an apparatus which, from the position coordinates, determines the colour and opacity value of the transfer function at the point with this scalar value. Finally, a further display apparatus is suitably provided which reproduces the structure data resulting from the volume data sets, preferably as a scalar value depiction with non-depicted location coordinate linking, further the imaging of the scanned structure(s) thereby acquired. Appropriate setting means are provided for selection of the imaging of the scanned structures. A computer is provided for data processing purposes. One advantageous embodiment is a personal computer, e.g. even a laptop.

The device preferably includes an apparatus which carries out classification of the voxels using different colour data values.

The invention is explained in more detail hereinunder with the aid of exemplified embodiments and the drawing in which:

FIG. 1 illustrates a display of a tooth (from left to right: automatic; interactive selection of dentine, dentine boundary, enamel, enamel boundary and nerve cavity, in each case with associated imaging of the transfer function,

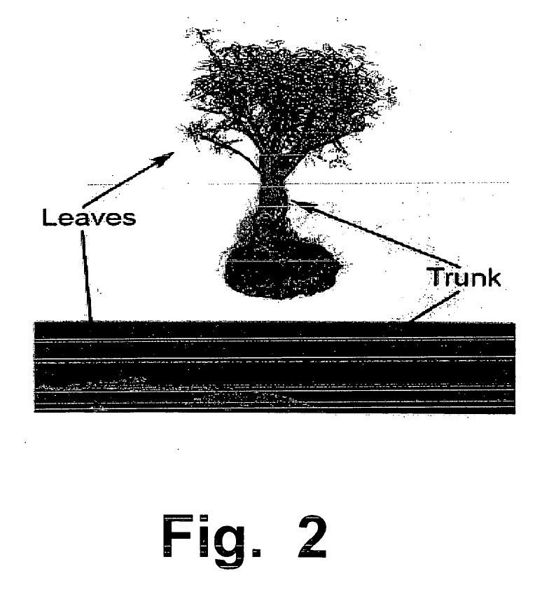

FIG. 2 illustrates a display of a bonsai tree,

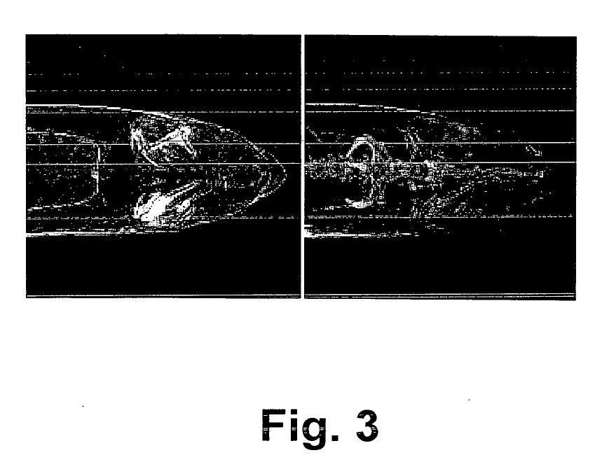

FIG. 3 illustrates a display of a carp, wherein the right view shows the result of the additional use of the region-growing method,

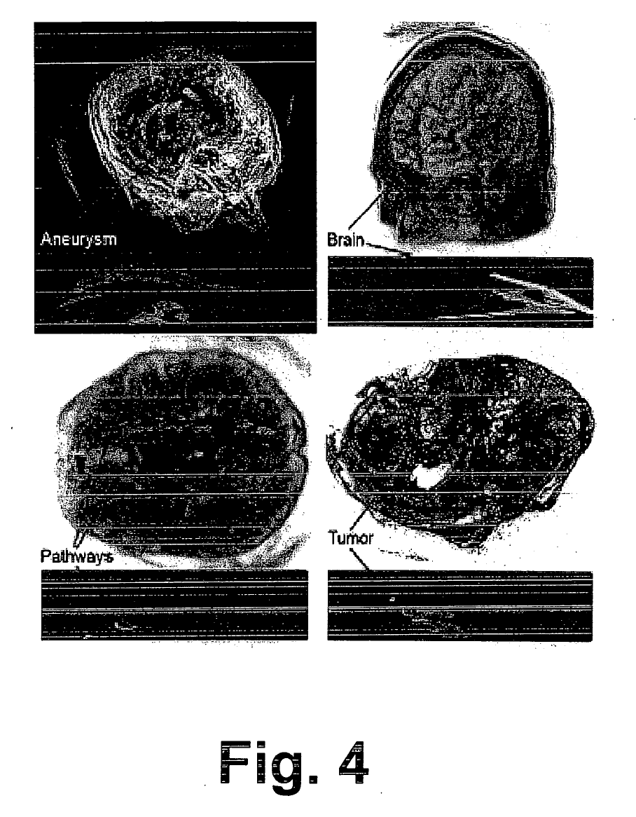

FIG. 4 illustrates four further practical display examples, and

FIG. 5 shows a functional diagram which shows the local resolution of the scanned structures.

Firstly, with the aid of an e.g. two-dimensional transfer function and by means of scalar values s and t, the associated opacity value F (s, t) for the imaging of scalar volume data sets of object structures is determined. In so doing an attempt is made to separate the illustrated objects. In this case advantage is taken of the fact that each object is spatially determined by its position. By means of the transfer function definite features should now be spatially allocated to definite colours in the transfer function, i.e. the correspondence between the scalar values and the objects should be found. In particular, all entries H in the transfer function which relate to the same position in the volume data set should be allocated the same colour. For this purpose, in one preprocwssing step for pi (s, t), i=1 . . . n positions of n voxels for an entry H (s, t)=n of the histogram, the centroid b (s, t) is calculated for each volume and the spatial variance of the voxels is determined with the aid of the deviations of the voxel positions pi (s, t) from the centroid b. A reference tuple T0 (s, t) is assumed and, by determining a region with a radius r, it is assumed that all tuples T with ∥ b(T)−b(T0) ∥<r belong to the same feature. In order to determine whether a value tuple T (s, t) belongs to the same feature as a reference tuple T0 (s, t) the following is used as a measure of the spatial correspondence therefor
N(T;T0)=∥b(T0)−b(T0)∥+|v(T0)−v(T)|

On the basis of this norm all entries of the transfer functions are classified into groups which belong to the same feature provided no time-consuming segmentation needs to be carried out. The determination of the radius about the reference point, which is incorporated into the resolution of the structure segmentation, is the single parameter which is selected manually. Each group entry T is then allocated an emission value. All entries which belong to the same group as the reference tuple are allocated a specific colour. The complex shape of the object structures can be recognised automatically by reason of the spatial information contained in the transfer function.

This is illustrated by means of the example of the depiction of a tooth in FIG. 1, wherein six images are shown (with the aid of a data set from Pfister H., Lorensen W., Bajaj C., Kindlmann G., Schroeder W., Sobierajski Avila L., Martin K., Machiraju R., Lee J.: Visualization Viewpoints, “The Transfer Function Bake-Off”, IEEE Computer Graphics and Applications 21, 3 (2001)). In FIG. 1 the individual images comprise, at the top, the respective actual depiction and, at the bottom, the associated transfer function, while at the far left the original two-dimensional histogram is shown. At the top right the intensity peak values of the depiction of the scattering width are clearly split into uniformly coloured regions which correspond to different materials and the boundaries thereof. Each illustrated feature has been selected by selecting the correspondingly accentuated zone below each image.

The automatic display carried out by the method in accordance with the invention is shown, as mentioned, on the far left of FIG. 1. The dentine, enamel and the boundary between the two materials have been coloured automatically. The maximum radius of the feature r is the only value which is set manually for imaging purposes.

FIG. 2 shows the image of a bonsai tree which is based on a data set of Stefan Roettger, The Volume Library, 2004. The illustration of the leaves has been allocated the colour green and the illustration of the trunk has been allocated the colour brown. In the image the so-called pseudo-shading technique has been applied for the purpose of emphasizing the object depiction or for better feature discrimination. This exploits the fact that at the object boundaries the scalar values rapidly decrease. By lowering the emission of the lowest scalar values of the object, its silhouette appears dark since the opacity becomes dominant, and an image is produced as if the object had been illuminated by means of a spot light. Specifically, in the transfer function illustrated at the bottom of FIG. 2 the class which is luminescing strongly in green and the brown class have been selected and it has been determined that depiction should be effected by means of pseudo-shading. For each class the scalar value range has been determined and then, for each class, emission has been diminished over the scalar value range using a linear step (ramp).

Further examples of the display of object structures in accordance with the invention are described hereinunder. As already mentioned, in the reconstruction of the object structures problems arise under certain circumstances in allocating depiction parameters to actual objects or classes insofar as the allocation is not clear. This is always the case when a plurality of objects have the same material properties (e.g. bone). By the addition of further differentiation criteria (fat proportion, tissue or muscle structures) it is possible e.g. to make visible a bone fracture in a bone structure which cannot be depicted by a conventional transfer function alone.

The so-called region-growing method is preferably used in this case, being applied to pixels with opacity not equal to zero and using similarities in adjacent pixels to find the object. If the neighbouring point of a considered point has similar features it is ascribed to the object of the point being considered. By successive procedure, areas thus associated are produced, starting with substructures or starting points, and partial structures of objects are detected.

FIG. 3 shows the image of a carp, the region-growing method having been used therein. The adaptation to the method in accordance with the invention consisted of splitting the object structures into the partial structures with the aid of spatial connectivity. This specific type of segmentation is extraordinarily quick since for each voxel it is only necessary to resort to two-dimensional opacity depiction (map). Each detected segment is allocated any identification mark (tag) which determines the colour toning value of the segment. With the aid of the rendering view a segment is selected using its colour toning value in precisely the manner effected with the aid of the transfer function in relation to the colouring. In the left-hand view of FIG. 3 the bones of the carp have been selected for viewing by means of the transfer function, wherein for the purpose of better orientation the skin is shown in a light colour (white). The right-hand view of FIG. 3 shows the bones of the carp segmented using the region-growing method. Each segment received a different colour value. The orange-coloured backbone has then been selected and accentuated. The prominence of the backbone has thus become clearly visible. Otherwise it would be concealed by the cranial bone of the head.

Further practical examples of the application of the method in accordance with the invention are shown by the views of FIG. 4.

An aneurysm is shown at the top left in FIG. 4, being shown as a red smudge of blood. This corresponds to the tiny red speck at the bottom of the illustration of the transfer function (see arrow). The small brown region thereabove maps to the inside of the artery in the brain. The red speck would be very difficult to locate without application of the transfer function in accordance with the invention since only the smallest spatial offset leads to selection of the arteries. By means of the transfer function in accordance with the invention the shape of the red speck is automatically established. The ability to see aneurysms immediately by means of the method in accordance with the invention has been confirmed on a large number of specimens.

For reasons of measuring technology, depiction of the cranium using MRI data is not satisfactory per se since its imaging is effected on almost the same scalar values as air. The separate display of the brain, cranium and tissue thus constitutes a problem. In order to solve this problem, instead of the standard 2D transfer function with scalar values and gradients, a transfer function based on the TI and proton density (PD) -weighted response of the MRI scanner has been used.

The top right of FIG. 4 shows the regions of the cranium and brain accentuated and depicted in accordance with the invention. The depiction can be carried out without much effort but should not be regarded as a replacement for true brain segmentation.

The bottom left of FIG. 4 shows the imaging of nerve tracts using diffusion tensor imaging (DTI). In a standard manner, during scanning, tracts are followed along the largest eigenvector of the tensor field and are discriminated from nerve cells by the incorporation of so-called fractional anisotropy of the diffusion tensor. Values with higher anisotropy are characteristic (white matter) of the tracts (pathways), while nerve cells have lower anisotropy (grey matter). For display purposes in the embodiment in accordance with the invention a two-dimensional transfer function based on the scalar values and fractional anisotropy was used. In the image shown, the tracts correspond to a characteristic region in the upper middle of the transfer function. A further characteristic region is the ventricle in the lower right region of the transfer function.

At the bottom right in FIG. 4 a practical example for the display of multi-modal data is shown. While, by means of MRI volume data sets, a tumour can be rendered visible, e.g. a CT scanner provides the depiction of the cranium required to plan a surgical operation. For the propose of superimposing cross-sections, registration of the two multi-modal data sets is carried out. By means of a two-dimensional transfer function in accordance with the invention, which is based on the two scanning modes, the tumour and the bone surrounding it can be selected immediately.

To summarise: In the case of the depiction method in accordance with the invention and with the aid of the transfer function each entry into this transfer function is determined with the aid of the spatial positions of the voxels with the associated scalar value. By virtue of the allocation instruction or the transfer function containing the spatial coordinates a very suitable means is provided for displaying structures, the features and properties thereof in scalar, diff-usion tensor or multi-modal volume data sets. When a feature has a characteristic surface in the region of the transfer function the feature can be depicted quickly by selecting the corresponding class in the transfer function. The transfer function can therefore be set up quickly and automatically.

1. Method for depicting structures within volume data sets, wherein
each voxel is allocated a colour (RGB) and opacity (A) by means of an allocation instruction (F (s, t, . . . )) in dependence upon the scalar values (s, t, . . . ),
wherein:
for each scalar value (s, t, . . . ) the spatial position (x, y, z) of the voxels with this scalar value is determined and
from the position coordinates (x, y, z) the colour and opacity value (RGB, A) of the allocation instruction (F (s, t, . . . )) at the site (s, t, . . . ) is determined.

2. Method as claimed in claim 1, wherein the allocation instruction is a transfer function F (s, t, . . . ).

3. Method as claimed in claim 1, wherein classification is effected according to the spatial position, and each class is allocated a specific colour (RGB) and/or opacity (A).

4. Method as claimed in claim 1, wherein the centroid is selected as the spatial position.

5. Method as claimed in claim 1, wherein classification is carried out according to the variance or the privileged direction of the said spatial positions, and each class is allocated a colour and/or opacity.

6. Method as claimed in claim 4, wherein one or a plurality of classes are selected for the depiction.

7. Method as claimed in claim 1, characterised in that wherein a one-dimensional transfer function (F (s)) is used.

8. Method as claimed in claim 1, characterised in that wherein a multi-dimensional transfer function (F (s, t, . . . )) is used.

9. Method as claimed in claim 1, wherein for the purposes of noise reduction the number of voxels is increased by overscanning.

10-14. (canceled)

15. Computer program product for the implementation of a method as claimed in claim 1.

16. Device for carrying out the method as claimed in claim 1, comprising:
an apparatus which allocates a colour (RGB) and opacity (A) to each voxel by means of a transfer function (T) in dependence upon the scalar values (s, t, . . . ),
an apparatus which, for each scalar value (s, t, . . . ), determines the spatial position of the voxels with this scalar value, and
an apparatus which, from the position coordinates (x, y, z), determines the colour and opacity value (RGB, A) of the transfer function (T) at the point (s, t, . . . ).
